SEQUENCE LISTING (9 sequences)

SEQ ID NO: 1 - protein, 244 aa, Homo sapiens
QDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGEVDIGDAAYYFERDIKGESLFQGRGGLDLRVRGEPLQVERTLIYYLDEIPPKFSMKRLT

SEQ ID NO: 2 - protein, 323 aa, Homo sapiens
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGEVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 3 - protein, 115 aa, Homo sapiens
QDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRC

SEQ ID NO: 4 - protein, 129 aa, Homo sapiens
DELVRTHHILIDLRHRPTAGAFNHSDLDAELRRLFRERYRLHPKFVAAVHYEQPTIQIELRQNTSQKAAGEVDIGDAAYYFERDIKGESLFQGRGGLDLRVRGEPLQVERTLIYYLDEIPPKFSMKRLT

SEQ ID NO: 5 - protein, 119 aa, Homo sapiens
QVQLVQSGAEVKKPGASVKVSCKASGFTFSSSYISWLRQAPGQRLEWIAWIYAGTGGTSYNQKFTGKATLTVDTSASTAYMELSSLRSEDTAVYYCARHNPRYYAMDYWGQGTTVTVSS

SEQ ID NO: 6 - DNA, 357 bp, Homo sapiens
  source 1..357: mol_type = genomic DNA
caagtgcagctcgtccagtctggagctgaagtcaaaaagcctggggcttcagtgaaagtctcctgcaaggcttctggcttcaccttcagcagtagctatatcagttggttgaggcaggcccctggacagagacttgagtggattgcatggatttatgctggaactggcggaactagctataatcagaagttcacaggcaaggccacactgactgtagacacatccgccagcacagcctacatggaactcagcagcctgagatctgaggacactgccgtctattactgtgcaagacataaccctcgttactatgctatggactactggggccaaggaaccacagtcaccgtctcctca

SEQ ID NO: 7 - protein, 108 aa, Homo sapiens
DTQMTQSPSSLSASVGDRVTITCITSTDIDDDMNWYQQKPGKAPKLLISEGNTLRPGVPSRFSGSGYGTDFTFTISSLQPEDIATYYCLQSDNLPYTFGGGTKVEIKR

SEQ ID NO: 8 - DNA, 324 bp, Homo sapiens
  source 1..324: mol_type = genomic DNA
gacacccagatgacccagtctccaagctccctgtccgccagcgtgggagatagagtcaccatcacatgcatcaccagcactgatattgatgatgatatgaactggtaccagcagaagccagggaaagctcctaagctcctgatttcagaaggcaatactctgcgccctggagtcccatcccgattctccggcagtggctatggaacagattttacctttacaattagctccctgcagccagaagatattgcaacctactactgtttgcaaagtgataacctgccctacaccttcggaggggggaccaaagtcgaaatcaaacgg

SEQ ID NO: 9 - protein, 8 aa, synthetic construct
  REGION 1..8: note = Flag epitope
DYKDDDDK

(19) United States
(12) Patent Application Publication
(10) Pub. No.: US 2025/0302877 A1
(43) Pub. Date: Oct. 2, 2025

(54) ANTI-ACTIVATED TROP-2 CHIMERIC ANTIGEN RECEPTOR CONSTRUCTS FOR USE IN CANCER THERAPY

(71) Applicant: MEDITERRANEA THERANOSTIC S.R.L., Lanciano (IT)

(72) Inventor: Saverio ALBERTI, Lanciano (IT)

(73) Assignee: MEDITERRANEA THERANOSTIC S.R.L., Lanciano (IT)

(21) Appl. No.: 18/865,587

(22) PCT Filed: May 29, 2023
(86) PCT No.: PCT/EP2023/064339

§ 371 Date: Nov. 13, 2024

(30) Foreign Application Priority Data
May 30, 2022 (IT) 102022000011390

(51) Int. Cl. A61K 35/17; A61K 40/11; A61K 40/15; A61K 40/31; A61K 40/42; A61K 45/06; C07K 16/30

(52) U.S. Cl. CPC A61K 35/17; A61K 40/11; A61K 40/15; A61K 40/31; A61K 40/4202; A61K 45/06; C07K 16/30; A61K 2239/13; A61K 2239/21; C07K 2317/24; C07K 2317/56; C07K 2317/622

(57) ABSTRACT

Chimeric antigen receptors (CARs) expressible by cells of the immune system such as T cells or NK cells (CAR-T and CAR-NK). These CARs comprise a fragment of scFv antibody directed against a specific antigen of tumor tissues, the tumor activated Trop-2 antigen wherein said activated Trop-2 is the Trop-2 molecule proteolytically cleaved between R87 and T88. The invention also involves the use of CAR constructs for use in anti-tumor cell therapy.

### STATE OF THE ART

The development of anticancer immunotherapy represents one of the main challenges in today's oncology. Chimeric antigen receptor (CAR)-T or NK cell therapy is a promising T/NK cell therapeutic engineering practice. This is based on artificial CARs that target specific tumor-associated antigens and can directly connect the antibody-antigen recognition with the cytotoxic activities of immune effector cells. Immune cells are isolated from the patient's peripheral blood, a procedure known as leukapheresis, to be engineered in vitro to express a specific CAR, and once infused back in the patient they can trigger an immune response against the corresponding target antigen and cells expressing that antigen.

Genetic modification of peripherally derived T cells with CAR was developed from the concept of adoptive immunotherapy using tumor-infiltrating lymphocytes (TILs), whose T cell receptors (TCRs) recognize tumor-associated antigens. CAR-T recognize the target antigen through immunoglobulin antigen-binding regions, hence bypassing the need for MHC presentation.

CARs typically comprise an antibody fragment, such as a scFv or Fab fragment, incorporated in a fusion protein that also comprises additional components, such as a CD3-ζ or CD28 transmembrane domain and selective T-cell activating moieties, including the endodomains of CD3-ζ, CD28, OX40, 4-IBB, Lek and/or ICOS (FIG. 1).

CAR constructs have been correspondingly used to direct the activity of natural killer (NK) cells, which are easily isolated from donors by a simple blood draw. Cancer cells that do not cause inflammation tend to be treated as self by the immune system and do not efficiently stimulate a T cell response. NK cells are more readily activated to produce several cytokines, including tumor necrosis factor α, interferon γ and IL-10 (Jiang, Zhang et al. 2014). The activation of NK cells leads to the gradual formation of immune effectors cells, such as dendritic cells, macrophages, and neutrophils, which consequently facilitate antigen-specific T and B cell responses. NK cell-mediated tumor cell lysis involves distinct receptors, including NKp44, NKp46, NKG2D, NKp30 and DNAM. Malignant cells usually express NKG2D in addition to ULBP and MICA (Romanski, Uherek et al. 2016).

Like T cells, NK cells can be transfected with CAR constructs, used to induce an anti-cancer immune response. NK cells do not require HLA matching; therefore, they can be used as allogeneic effector cells (Hermanson and Kaufman 2015). The lack of endogenous antigen specificity for targeting cells to be killed, allows NK cells, unlike T cells, to be easily re-directed by CARs. The cell-targeting scFv or Fab may be linked via a transmembrane domain to one or more intracellular signalling domains to induce lymphocyte activation. Signalling domains used with CAR-NK cells have included CD3-ζ, CD28, 4-IBB, DAPIO and OX40. NK cell lines, capable of cytotoxic activity, e.g., NK-92, NKG, YT, NK-YS, HANK-I, YTS, NKL, have also been used as CAR construct recipient. A great advantage of these cell lines is their essentially unlimited availability as ‘off-the-shelf’ reagents for injection in multiple, unrelated patients.

The clinical efficacy of CAR-T or NK cells in haematological malignancies is rarely replicated in solid tumours. Haematological malignancies are often circulating/disseminated, and as such are lacking many of the physical immunosuppressive factors that hamper adoptively transferred cells from reaching solid tumours. Furthermore, target antigens that are present on haematological cancers are often homogenous and expressed in a majority if not all the tumor cells. In contrast, target antigens on solid tumours are often heterogeneous, differing not just within one tumor but also between primary and metastatic tumours. CAR-T-cell therapy for solid tumours therefore faces multiple hurdles.

Trop-2 is a cell-surface signal transducer (Basu, Goldenberg et al. 1995, Ripani, Sacchetti et al. 1998), that induces an intracellular calcium spike after cross-linking with antibodies (Ripani, Sacchetti et al. 1998). Trop-2 was shown to drive cancer development and progression through interaction and regulation of expression and activity of cyclin D1, ERK, NFkB (Guerra, Trerotola et al. 2008, Guerra, Trerotola et al. 2013, Trerotola, Cantanelli et al. 2013), FAK, Rac1 and integrins (Trerotola, Li et al. 2012, Trerotola, Jernigan et al. 2013).

Trop-2 heavily modulates the E-cadherin/β-catenin pathway in metastasis. Overexpression of Trop-2 causes the release of E-cadherin from the cytoskeleton, for loss of cell-cell adhesion, and activation of β-catenin transcription and anti-apoptotic signaling, with the induction of a malignant phenotype. This global, Trop-2/E-cadherin/β-catenin-driven pro-metastatic program profoundly impacts on the survival of patients bearing breast, colon, ovary, uterus and stomach tumors (Guerra, Trerotola et al. 2021, Trerotola, Guerra et al. 2021).

High levels of expression of Trop-2 occur in multiple cancer types (Alberti, Miotti et al. 1992, Stepan, Trueblood et al. 2011, Trerotola, Cantanelli et al. 2013, Guerra, Trerotola et al. 2021, Trerotola, Guerra et al. 2021, Guerra, Relli et al. 2022), supporting the use of Trop-2 as a therapy target. However, Trop-2 was also shown to be highly expressed in several normal tissues (Alberti, Miotti et al. 1992, Stepan, Trueblood et al. 2011, Trerotola, Cantanelli et al. 2013, Guerra, Trerotola et al. 2021, Trerotola, Guerra et al. 2021, Guerra, Relli et al. 2022), including epidermis, endometrium, oesophagus, tonsil, lung, kidney, salivary glands and breast (Guerra, Relli et al. 2022). This was expected to cause toxicity in patients treated with anti-Trop-2 drugs, because of on-target/off-tumor recognition.

The authors of the present invention have discovered that processing mediated by ADAM10 triggers Trop-2 activation (Guerra, Trerotola et al. 2021, Trerotola, Guerra et al. 2021). Trop-2 activation correlates with the cleavage in the first thyroglobulin domain loop of the Trop-2 extracellular domain, between R87-T88. Molecular modeling indicated that this induces a profound rearrangement of Trop-2 structure, consistent with a regulatory function. The role of Trop-2 processing for metastatic spreading was shown, utilizing in vitro and in vivo systems, including colon cancer xenografts.

Of note, no processed Trop-2 was detected in normal human keratinocytes, or other normal tissues (tongue, urinary bladder, heart, salivary gland, mammary gland, skin, kidney, parotid gland, esophagus, lung, liver and pancreas) from humans and primates. On the other hand, the presence of proteolytic processing was shown in breast, ovarian and colon cancer (Guerra, Trerotola et al. 2021, Trerotola, Guerra et al. 2021).

WO2010089782 describes anti-Trop-2 murine monoclonal antibodies (mAbs), among them the 2G10 mAb (hybridoma cell line deposited in the Advanced Biotechnology Center-ICLC of Genoa with the number PD 08020), which are active against the growth of multiple types of tumors such as for example Colo-205, HCT-116 and HT29 colon cancers; SKOV ovarian cancer; SKBR3 and MDA MB468 breast cancer.

WO2016087651 describes anti-Trop-2 human mAbs, which were designed to reduce immune responses against the injected mAb in patients bearing target cancers.

A fundamental need exists for improved design of CAR-T and CAR-NK constructs, and CAR strategies, with better efficacy and decreased systemic toxicity.

### DESCRIPTION OF THE INVENTION

The solution to the actual problem has been surprisingly found by the author of the present invention in producing a chimeric antigen receptor (CAR) which comprises an activated-Trop-2 binding domain wherein said activated Trop-2 is the Trop-2 molecule proteolytically cleaved between R87 and T88.

Therefore, it forms an object of the present invention a CAR construct for binding to the cleaved-activated Trop-2 target in cancer cells, while sparing normal cells expressing wt, uncleaved Trop-2.

### DRAWINGS DESCRIPTION

FIG. 1: (A) the CAR construct, comprising CD3ζ as activatory and 4-1 BB and CD28 as co-stimulatory domains, a CD28 transmembrane domain (TM), a flag F and a Signal sequence S. (B) schematic representation of the expressed construct, showing the scFv linked through a transmembrane domain to an intracellular T-cell signalling domain, in particular CD3ζ, which is the primary activation domain for TCR mediated T-cell activation.

FIG. 2: Flow cytometry analysis of Jurkat cells transduced with anti-activated Trop-2 CAR wherein said activated Trop-2 is the Trop-2 molecule proteolytically cleaved between R87 and T88. Horizontal axis: fluorescence staining. Vertical axis: cell forward scatter, related to cell dimensions. Each dot represents the reading for a single cell. Top panels: the CAR elements order is ‘light chain-linker-heavy chain’ (comparative); Bottom panel: the order is ‘heavy chain-linker-light chain’ (according to the invention). (A) control profile of unstained cells. (B, C) anti-FLAG-Alexa488-antibody binding a synthetic tag, the Flag, added to the CAR construct. The ovals encompass positive CAR-expressing cells.

### DETAILED DESCRIPTION

Unless specifically defined herein, all technical and scientific terms used have the same meaning as commonly understood by a skilled person in the fields of gene therapy, biochemistry, genetics, and molecular biology.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, Third Edition, (Sambrook et al, 2001, Cold Spring Harbor, New York: Cold Spring Harbor Laboratory Press).

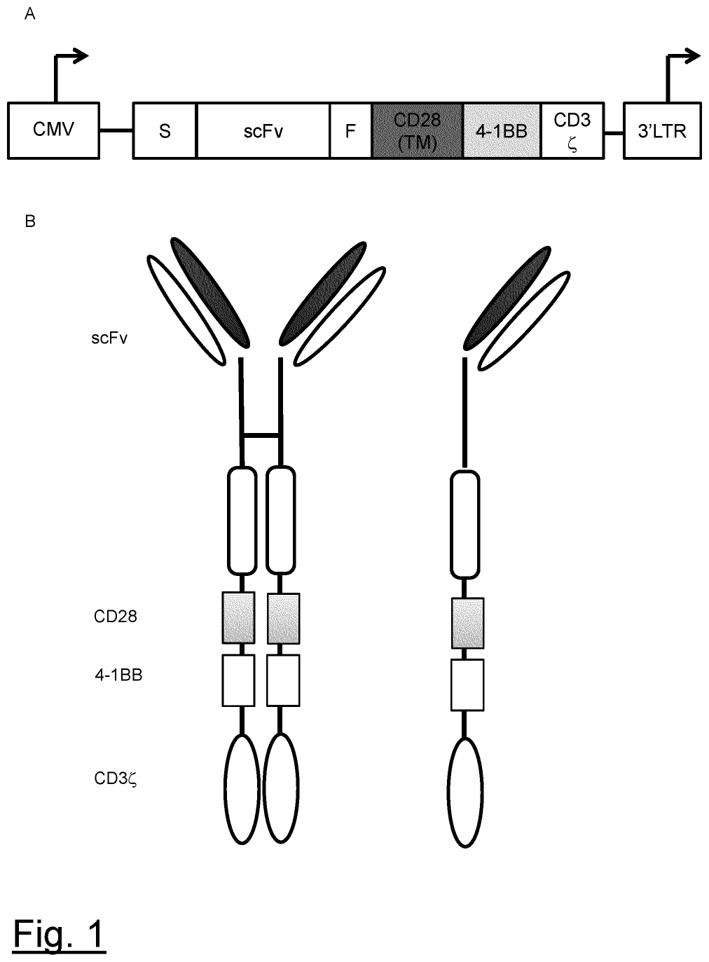

It forms an object of the present invention a Trop-2-specific chimeric antigen receptor (Trop-2-CAR) comprising, according to FIG. 1, an extracellular ligand-binding domain comprising VH and VL from a monoclonal anti-Trop-2 antibody, a transmembrane domain, a cytoplasmic domain.

In an embodiment, said anti-Trop-2 antibody is an antibody specific for the activated Trop-2 wherein said activated Trop-2 is the Trop-2 molecule proteolytically cleaved between R87 and T88.

Anti-activated Trop-2 antibodies wherein said activated Trop-2 is the Trop-2 molecule proteolytically cleaved between R87 and T88 were generated using an immunogen comprising both the entire extracellular portion (SEQ ID NO: 1, corresponding to amino acids 31-274 of SEQ ID NO: 2) and single domains of the Trop-2 molecule (SEQ ID NO: 3, globular domain: amino acids 31-145 of SEQ ID NO: 1; SEQ ID NO: 4 “stem”: amino acids 146-274 of SEQ ID NO: 2). These were produced in their native folding in human 293 transformed kidney epithelial cells and MCF-7 breast adenocarcinoma, and murine L fibrosarcoma and NS-0 myeloma, in insect Sf9 and yeast cells. Expression vectors for production were generated using PCR amplification of Trop-2 coding sequences fused to tags for purification or immunogenicity enhancement. The PCR fragments were subcloned in the vectors described above and expressed in the corresponding hosts. The Trop-2 proteins were purified by affinity chromatography. BALB/c mice were subjected to multiple immunization cycles with the immunogen described above, following best procedures known in the art. Splenocytes from immunized mice were fused to Sp2/0 or NS-0 myeloma cells and corresponding hybridomas were obtained, according to the methods known in the art. The antibodies produced by the hybridomas thus obtained were screened for specific and differential reactivity towards the processed Trop-2 that is expressed by tumor cells. mAbs 1A9 and 11B4 have been selected for their ability to recognize and bind with high affinity only the tumor-specific processed form of Trop-2, and not the unprocessed Trop-2 found in normal tissues. Flow cytometry cross-competition experiments between 1A9, 1B4 and T16 mAbs on KM12SM/wtTrop-2 and KM12SM/vector transfectants demonstrated that 1A9 and 1B4 blocked each other's binding, thus indicating recognition of the same epitope. On the contrary, there is no competition of 1A9 and 1B4 against T16, demonstrating how the procedures here described have effectively allowed to obtain mAbs for an epitope that is different from the immunodominant one.

In an embodiment, said VH domain is the VH domain of the 1B4 anti-Trop-2 mAb, secreted by the hybridoma deposited with the International Depositary Authority (IDA): Interlab Cell Line Collection, IRCCS Ospedale Policlinico San Martino, Genova, Italy, and assigned accession number PD21005.

In an embodiment, said VH domain is the VH domain of the 1A9 anti-Trop-2 mAb, secreted by the hybridoma deposited with the International Depositary Authority (IDA): Interlab Cell Line Collection, IRCCS Ospedale Policlinico San Martino, Genova, Italy, and assigned accession number PD21006.

In an embodiment, said VL domain is the VL domain of the 1B4 anti-Trop-2 mAb, secreted by the hybridoma deposited with the International Depositary Authority (IDA): Interlab Cell Line Collection, IRCCS Ospedale Policlinico San Martino, Genova, Italy, and assigned accession number PD21005.

In an embodiment, said VL domain is the VL domain of the 1A9 anti-Trop-2 mAb, secreted by the hybridoma deposited with the International Depositary Authority (IDA): Interlab Cell Line Collection, IRCCS Ospedale Policlinico San Martino, Genova, Italy, and assigned accession number PD21006.

In an embodiment, said VH domain has the sequence shown as SEQ ID NO: 5 for protein, SEQ ID NO: 6 for DNA or a variant thereof having at least 80% sequence identity which retains the capacity to bind activated Trop-2 wherein said activated Trop-2 is the Trop-2 molecule proteolytically cleaved between R87 and T88.

SEQ ID NO: 5
QVQLVQSGAEVKKPGASVKVSCKASGFTFSSSYISWLRQAPGQRLEWIA
WIYAGTGGTSYNQKFTGKATLTVDTSASTAYMELSSLRSEDTAVYYCAR
HNPRYYAMDYWGQGTTVTVSS
SEQ ID NO: 6
CAAGTGCAGCTCGTCCAGTCTGGAGCTGAAGTCAAAAAGCCTGGGGCT
TCAGTGAAAGTCTCCTGCAAGGCTTCTGGCTTCACCTTCAGCAGTAGCT
ATATCAGTTGGTTGAGGCAGGCCCCTGGACAGAGACTTGAGTGGATTG
CATGGATTTATGCTGGAACTGGCGGAACTAGCTATAATCAGAAGTTCAC
AGGCAAGGCCACACTGACTGTAGACACATCCGCCAGCACAGCCTACAT
GGAACTCAGCAGCCTGAGATCTGAGGACACTGCCGTCTATTACTGTGCA
AGACATAACCCTCGTTACTATGCTATGGACTACTGGGGCCAAGGAACCA
CAGTCACCGTCTCCTCA

In an embodiment, said VL domain has the sequence shown as SEQ ID NO: 7 for protein, SEQ ID NO: 8 for DNA or a variant thereof having at least 80% sequence identity which retains the capacity to bind activated Trop-2 wherein said activated Trop-2 is the Trop-2 molecule proteolytically cleaved between R87 and T88.

SEQ ID NO: 7
DTQMTQSPSSLSASVGDRVTITCITSTDIDDDMNWYQQKPGKAPKLLISE
GNTLRPGVPSRFSGSGYGTDFTFTISSLQPEDIATYYCLQSDNLPYTFGG
GTKVEIKR
SEQ ID NO: 8
GACACCCAGATGACCCAGTCTCCAAGCTCCCTGTCCGCCAGCGTGGGA
GATAGAGTCACCATCACATGCATCACCAGCACTGATATTGATGATGATAT
GAACTGGTACCAGCAGAAGCCAGGGAAAGCTCCTAAGCTCCTGATTTCA
GAAGGCAATACTCTGCGCCCTGGAGTCCCATCCCGATTCTCCGGCAGT
GGCTATGGAACAGATTTTACCTTTACAATTAGCTCCCTGCAGCCAGAAG
ATATTGCAACCTACTACTGTTTGCAAAGTGATAACCTGCCCTACACCTTC
GGAGGGGGGACCAAAGTCGAAATCAAACGG

In an embodiment, the antibodies taught by the present invention are humanized or chimeric antibodies, where the murine constant region is substituted by human constant regions, or variants containing at least one of the CDRs of the variable region of the corresponding light and/or heavy chains, so that the antibody specificity for the target is maintained, possibly mutagenized to modify their affinity for the target.

In an embodiment, said two VH and VL domains are linked by a spacer. Preferably, said spacer is GlyGlyGlyGlySer, more preferably, said motif is repeated 4 times.

In an embodiment, said domains are linked in this manner: VH-spacer-VL.

In an embodiment, said CAR comprises activatory and co-stimulatory domains, in an embodiment, they are selected from CD28, 4-1BB or CD137, inducible T cell co-stimulator (ICOS) or CD278, OX40, or CD134, or a CD3ζ. Preferably they are a CD3ζ, CD28 and 4-1 BB.

In some of any such embodiments, the transmembrane portion of CD28 is human CD28.

In an embodiment, said CAR comprises a Flag epitope, which is recognized by an anti-Flag mAb, to reveal CAR expression at the cell surface and identify CAR-transduced cells. In an embodiment, said Flag epitope has the sequence motif DYKDDDDK (SEQ ID NO: 9), a highly specific artificial antigen which is recognized by anti-FLAG mAbs known in the art.

In an embodiment, said CAR comprises a CD8 alpha signal sequence. In an embodiment, said CAR comprises a CD8 alpha hinge domain.

With reference to the schematic diagram of FIG. 1A, in a preferred embodiment said construct comprises: the CD3ζ activator domain, the two co-stimulatory domains, being CD28 and 4-1 BB, a Flag F, a Signal sequence S, the variable region of an anti activated-Trop-2 antibody wherein said activated Trop-2 is the Trop-2 molecule proteolytically cleaved between R87 and T88.

In a second embodiment, it is here claimed a cell, T or NK, which expresses a CAR according to the present description.

With reference to FIG. 1B, once expressed by a T cell, the construct according to the present invention shows the extracellular scFv portion linked through a transmembrane domain to the intracellular signaling domain, represented by the CD28, 4-1 BB co-stimulatory domains and the CD3ζ activatory domain.

In a third embodiment, it is here claimed a method for making a cell according to the present description, which comprises the step of introducing a nucleic acid sequence according to the instant description into a cell ex vivo. Said cell is preferably a T or a NK cell.

In a fourth embodiment, it is here claimed a pharmaceutical composition which comprises a plurality of cells according to the instant description.

In a fifth embodiment, it is here claimed said pharmaceutical composition for use in a method for treating a disease in a subject. In an embodiment, said disease is a tumour.

In a further embodiment, it is here described a method of eliciting a regulated immune response to a disease comprising administering to the patient a cell of the immune system such as a T cell or a NK cell that has been transfected with a CAR according to the present invention.

The authors of the present invention surprisingly demonstrated that CAR T or CAR NK cells expressing the V-genes of an anti Trop-2 antibody once administered to a subject are capable to induce an immune response against Trop-2-expressing target cells, as binding of CARs on the surface of transduced T cells or NK cells to antigens expressed by a target cell activates the transduced T or NK cell.

Following are examples that illustrate procedures for practicing the invention. These examples should not be construed as limiting.

### EXAMPLES

### Example 1

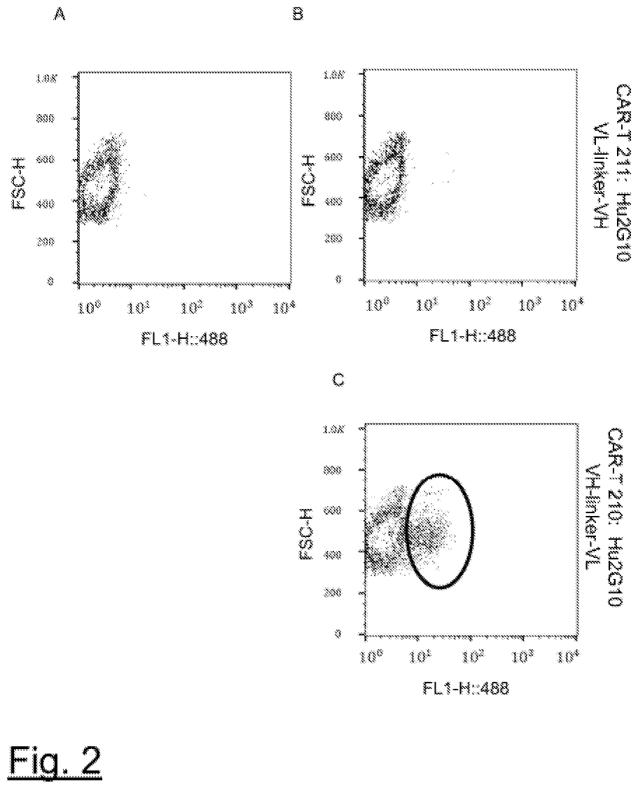

Jurkat cells were transduced with anti-activated Trop-2 CAR, wherein said activated Trop-2 is the Trop-2 molecule proteolytically cleaved between R87 and T88 wherein said VH has the sequence shown as SEQ ID NO: 5 for protein, SEQ ID NO: 6 for DNA and VL has the sequence shown as SEQ ID NO: 7 for protein, SEQ ID NO: 8 for DNA and the linker consists of 4 repetitions of the GlyGlyGlyGlySer motif. In a first embodiment (control) the CAR elements are VL-linker-VH. In a second embodiment, according to the invention, the CAR elements are VH-linker-VL. Cells were then analyzed by FACS after staining with an anti-FLAG-Alexa488 antibody (FIG. 2B-C). Unstained cells were also analyzed as control. Only the VH-linker-VL CAR elements showed fluorescence staining. i.e., CAR expression at the membrane level.

### REFERENCES

Alberti, S., S. Miotti, M. Stella, C. E. Klein, M. Fornaro, S. Ménard and M. I. Colnaghi (1992). “Biochemical characterization of Trop-2, a cell surface molecule expressed by human carcinomas: formal proof that the monoclonal antibodies T16 and MOv-16 recognize Trop-2.” Hybridoma 11: 539-535.
Basu, A., D. M. Goldenberg and R. Stein (1995). “The epithelial/carcinoma antigen EGP-1, recognized by monoclonal antibody RS7-3G11, is phosphorylated on serine 303.” Int J Cancer 62(4): 472-479.
Guerra, E., V. Relli, M. Ceci, R. Tripaldi, P. Simeone, A. L. Aloisi, L. Pantalone, R. La Sorda, R. Lattanzio, A. Sacchetti, K. Havas, S. Guarnieri, D. Vergara, I. Fournier, M. Salzet, N. Tinari, M. Piantelli, M. Trerotola and S. Alberti (2022). “Trop-2, Na+/K+ ATPase, CD9, PKCα, cofilin assemble a membrane signaling super-complex that drives colorectal cancer growth and invasion.” Oncogene 41(12): 1795-1808.
Guerra, E., M. Trerotola, A. L. Aloisi, R. Tripaldi, G. Vacca, R. La Sorda, R. Lattanzio, M. Piantelli and S. Alberti (2013). “The Trop-2 signalling network in cancer growth.” Oncogene 32: 1594-1600.
Guerra, E., M. Trerotola, R. Dell' Arciprete, V. Bonasera, B. Palombo, T. El-Sewedy, T. Ciccimarra, C. Crescenzi, F. Lorenzini, C. Rossi, G. Vacca, R. Lattanzio, M. Piantelli and S. Alberti (2008). “A bi-cistronic CYCLIN D1-TROP2 mRNA chimera demonstrates a novel oncogenic mechanism in human cancer.” Cancer Res 68(19): 8113-8121.
Guerra, E., M. Trerotola, V. Relli, R. Lattanzio, R. Tripaldi, G. Vacca, M. Ceci, K. Boujnah, V. Garbo, A. Moschella, R. Zappacosta, P. Simeone, R. de Lange, U. H. Weidle, M. T. Rotelli, A. Picciariello, R. Depalo, P. Querzoli, M. Pedriali, E. Bianchini, D. Angelucci, G. Pizzicannella, C. Di Loreto, M. Piantelli, L. Antolini, X.-F. Sun, D. F. Altomare and S. Alberti (2021). “Trop-2 induces ADAM10-mediated cleavage of E-cadherin and drives EMT-less metastasis in colon cancer.” Neoplasia 23(9): 898-911.
Hermanson, D. L. and D. S. Kaufman (2015). “Utilizing chimeric antigen receptors to direct natural killer cell activity.” Front Immunol 6: 195.
Jiang, H., W. Zhang, P. Shang, H. Zhang, W. Fu, F. Ye, T. Zeng, H. Huang, X. Zhang, W. Sun, D. Man-Yuen Sze, Q. Yi and J. Hou (2014). “Transfection of chimeric anti-CD138 gene enhances natural killer cell activation and killing of multiple myeloma cells.” Mol Oncol 8(2): 297-310.
Ripani, E., A. Sacchetti, D. Corda and S. Alberti (1998). “The human Trop-2 is a tumor-associated calcium signal transducer.” Int. J. Cancer 76: 671-676.
Romanski, A., C. Uherek, G. Bug, E. Seifried, H. Klingemann, W. S. Wels, O. G. Ottmann and T. Tonn (2016). “CD19-CAR engineered NK-92 cells are sufficient to overcome NK cell resistance in B-cell malignancies.” J Cell Mol Med 20(7): 1287-1294.
Stepan, L. P., E. S. Trueblood, K. Hale, J. Babcook, L. Borges and C. L. Sutherland (2011). “Expression of Trop2 Cell Surface Glycoprotein in Normal and Tumor Tissues: Potential Implications as a Cancer Therapeutic Target.” J Histochem Cytochem.
Trerotola, M., P. Cantanelli, E. Guerra, R. Tripaldi, A. L. Aloisi, V. Bonasera, R. Lattanzio, R. de Lange, U. H. Weidle, M. Piantelli and S. Alberti (2013). “Up-regulation of Trop-2 quantitatively stimulates human cancer growth.” Oncogene 32 222-233.
Trerotola, M., E. Guerra, Z. Ali, A. L. Aloisi, M. Ceci, P. Simeone, A. Acciarito, P. Zanna, G. Vacca, A. D'Amore, K. Boujnah, V. Garbo, A. Moschella, R. Lattanzio and S. Alberti (2021). “Trop-2 cleavage by ADAM10 is an activator switch for cancer growth and metastasis.” Neoplasia 23(4): 415-428.
Trerotola, M., D. Jernigan, Q. Liu, J. Siddiqui, A. Fatatis and L. Languino (2013). “Trop-2 promotes prostate cancer metastasis by modulating β1 integrin functions.” Cancer Res. 73(10): 3155-3167.
Trerotola, M., J. Li, S. Alberti and L. R. Languino (2012). “Trop-2 inhibits prostate cancer cell adhesion to fibronectin through the β1 integrin-RACK1 axis.” J Cell Physiol 227(11): 3670-3677.

1. A Trop-2-specific chimeric antigen receptor (CAR) having a structure comprising an extracellular ligand-binding domain comprising a heavy chain variable region (VH) and a light chain variable region (VL) from a monoclonal anti-Trop-2 antibody, a transmembrane domain, a cytoplasmic domain.

2. The CAR according to claim 1, wherein said antibody is a humanised 2G10 antibody.

3. The CAR according to claim 1, wherein said antibody specifically recognises activated Trop-2 wherein said activated Trop-2 is the Trop-2 molecule proteolytically cleaved between R87 and T88.

4. The CAR according to claim 3, wherein said VH is selected from the group consisting of the VH domain of the 1B4 anti-Trop-2 mAb, secreted by the hybridoma deposited with the International Depositary Authority (IDA): Interlab Cell Line Collection, IRCCS Ospedale Policlinico San Martino, Genova, Italy, and assigned accession number PD21005 and the VH domain of the 1A9 anti-Trop-2 mAb, secreted by the hybridoma deposited with the International Depositary Authority (IDA): Interlab Cell Line Collection, IRCCS Ospedale Policlinico San Martino, Genova, Italy, and assigned accession number PD21006.

5. The CAR according to claim 3, wherein said VL is selected from the group consisting of the VL domain of the 1B4 anti-Trop-2 mAb, secreted by the hybridoma deposited with the International Depositary Authority (IDA): Interlab Cell Line Collection, IRCCS Ospedale Policlinico San Martino, Genova, Italy, and assigned accession number PD21005 and the VL domain of the the 1A9 anti-Trop-2 mAb, secreted by the hybridoma deposited with the International Depositary Authority (IDA): Interlab Cell Line Collection, IRCCS Ospedale Policlinico San Martino, Genova, Italy, and assigned accession number PD21006.

6. The CAR according to claim 3, wherein said VH has the sequence shown as SEQ ID NO: 5 for protein, SEQ ID NO: 6 for DNA or a variant thereof having at least 80% sequence identity which retains the capacity to bind activated Trop-2 wherein said activated Trop-2 is the Trop-2 molecule proteolytically cleaved between R87 and T88.

7. The CAR according to claim 3, wherein said VL has the sequence shown as SEQ ID NO: 7 for protein, SEQ ID NO: 8 for DNA or a variant thereof having at least 80% sequence identity which retains the capacity to bind activated Trop-2 wherein said activated Trop-2 is the Trop-2 molecule proteolytically cleaved between R87 and T88.

8. The CAR according to claim 3, wherein said VL has the sequence shown as SEQ ID NO: 5 for protein, SEQ ID NO: 6 for DNA.

9. The CAR according to claim 3, wherein said VH has the sequence shown as SEQ ID NO: 7 for protein, SEQ ID NO: 8 for DNA.

10. The CAR according to claim 3, wherein said VL has the sequence shown as SEQ ID NO: 5 for protein, SEQ ID NO: 6 for DNA and said VH has the sequence shown as SEQ ID NO: 7 for protein, SEQ ID NO: 8 for DNA, said VH and VL being linked by a spacer.

11. The CAR according to claim 10, said spacer being GlyGlyGlyGlySer.

12. The CAR according to claim 11, said motif being repeated 4 times.

13. The CAR according to claim 10, said domains being linked in this manner: VH-spacer-VL.

14. A T-cell (CAR-T) or NK cell (CAR-NK) transduced with a CAR according to claim 1.

15. A pharmaceutical composition comprising CAR-T or CAR-NK cells according to claim 14.

16. The CAR according to claim 1 formulated, for use in the treatment of a disease.

17. The CAR according to claim 16, wherein said disease is a solid cancer expressing activated Trop-2 wherein said activated Trop-2 is the Trop-2 molecule proteolytically cleaved between R87 and T88.

18. The CAR for use according to claim 16, wherein said CAR is used together with at least one additional active agent selected from the group comprising: (i) an interferon; (ii) a checkpoint inhibitor antibody; (iii) an antibody-drug conjugate (ADC); (iv) an anti-HLA-DR antibody; (v) an anti-CD74 antibody.

19. A method for treating a disease in a subject comprising administering the Trop-2-specific chimeric antigen receptor (CAR) to a subject in need thereof.

20. The method of claim 19, wherein the disease is cancer.
